(19) United States
(12) Patent Application Publication
(10) Pub. No.: US 2026/0098045 A1
(43) Pub. Date: Apr. 9, 2026

(54) CRYSTALLINE FORM OF (3S,7S,10R,13R)-13-BENZYL-20-FLUORO-7-ISOBUTYL-N-(2-(3-METHOXY-1,2,4-OXADIAZOL-5-YL)ETHYL)-6,9-DIMETHYL-1,5,8,11-TETRAOXO-10-(2,2,2-TRIFLUOROETHYL)-1,2,3,4,5,6,7,8,9,10,11,12,13,14-TETRADECAHYDRO-[1]OXA[4,7,10,14]TETRAAZACYCLOHEPTADECINO[16,17-F]QUINOLINE-3-CARBOXAMIDE

(71) Applicant: Idorsia Pharmaceuticals Ltd, Allschwil (CH)

(72) Inventors: Charlyse HERRMANN, Allschwil (CH); Timo RAGER, Allschwil (CH)

(21) Appl. No.: 19/111,955

(22) PCT Filed: Sep. 14, 2023
(86) PCT No.: PCT/EP2023/075235

§ 371 Date: Mar. 14, 2025

(30) Foreign Application Priority Data
Sep. 15, 2022 (WO) PCT/EP2022/075606

(51) Int. Cl. C07D 498/04; A61K 31/4745; A61P 11/00

(52) U.S. Cl. CPC C07D 498/04; A61K 31/4745; A61P 11/00; C07B 2200/13

(57) ABSTRACT

The invention relates to crystalline form of (3S,7S,10R,13R)-13-benzyl-20-fluoro-7-isobutyl-N-(2-(3-methoxy-1,2,4-oxadiazol-5-yl)ethyl)-6,9-dimethyl-1, 5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide; processes for the preparation thereof, pharmaceutical compositions containing such crystalline form, pharmaceutical compositions prepared from such crystalline form, and their use as a medicament, especially as CFTR modulators.

The present invention relates to a novel crystalline form of (3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide (hereinafter also referred to as “COMPOUND”):

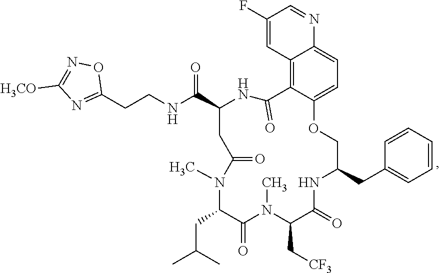 [SMILES: COc1noc(CCNC(=O)[C@@H]2CC(=O)N(C)[C@@H](CC(C)C)C(=O)N(C)[C@H](CC(F)(F)F)C(=O)N[C@H](Cc3ccccc3)COc3ccc4ncc(F)cc4c3C(=O)N2)n1]

processes for the preparation thereof, pharmaceutical compositions comprising said crystalline form, pharmaceutical compositions prepared from such crystalline form, and their use as CFTR modulators, especially for the treatment of cystic fibrosis. The invention further relates to said crystalline form as pharmaceutical in combination with one or more therapeutically active ingredients acting as CFTR modulator(s).

Cystic Fibrosis (CF; mucoviscidosis, sometimes also called fibrocystic disease of pancreas or pancreatic fibrosis) is an autosomal recessive genetic disease caused by a dysfunctional epithelial chloride/bicarbonate channel named Cystic Fibrosis Transmembrane Conductance Regulator (CFTR). CFTR dysfunction leads to dysregulated chloride, bicarbonate and water transport at the surface of secretory epithelia causing accumulation of sticky mucus in organs including lung, pancreas, liver and intestine and, as a consequence, multi-organ dysfunction. Most debilitating effects in CF are nowadays observed in the lung which—due to abnormal hydration of airway surface liquid, mucus plugging, impaired mucociliary clearance, chronic inflammation and infection—loses its functionality over time leading to death by respiratory failure (Elborn, 2016). Human CFTR is a multidomain protein of 1480 amino acids. Many different mutations causing CFTR dysfunction have been discovered in CF patients leading e.g. to no functional CFTR proteins (class I mutations), CFTR trafficking defects (class II mutations), CFTR regulation defects (also known as gating defects; class III mutations), CFTR conductance defects (class IV mutations), less CFTR protein either due to splicing defects (class V mutations) or due to reduced CFTR stability (class VI mutations), no CFTR protein due to mRNA instability (class VII mutations) (de Boeck, Acta Paediatr. 2020, 109(5):893-895). The CFTR2 database (http://cftr2.org; data retrieved 22.08.2022) currently contains information on 401 disease-causing mutations. By far the most common disease-causing mutation is the deletion of phenylalanine at position 508 (F508del; allele frequency 0.697 in the CFTR2 database), that leads to misfolding of the channel during synthesis at the endoplasmic reticulum, degradation of the misfolded protein and a resulting strongly reduced transport to the cell surface (class II mutation). The residual F508del-CFTR that is trafficked to the cell surface is functional, however less than wildtype CFTR, i.e. F508del-CFTR also harbours a gating defect (Dalemans, 1991). Ca 40% of all CF patients are homozygous for the F508del mutation while another ˜40% of patients are heterozygous for the F508del mutation and carry another disease-causing mutation from class I, II, III, IV, V, VI or VII. Such disease-causing mutations are considerably rarer with the class III G551D mutation (allele frequency 0.0210) and the class I G542X mutation (allele frequency 0.0254) and the class II N1303K mutation (allele frequency 0.0158) being the next most prevalent.

CF is currently treated by a range of drugs addressing the various organ symptoms and dysfunctions. Intestinal and pancreatic dysfunction are treated from diagnosis by food supplementation with pancreatic digestive enzymes. Lung symptoms are mainly treated with hypertonic saline inhalation, mucolytics, anti-inflammatory drugs, bronchiodilators and antibiotics (Elborn, 2016).

In addition to symptomatic treatments, CFTR modulators have been developed and approved for patients with certain CFTR mutations. These compounds directly improve CFTR folding and trafficking to the cell surface (CFTR correctors) or improve CFTR function at the cell surface (CFTR potentiators). Other types of modulators are still in the exploratory phase such as compounds that increase mRNA levels of (mutated) CFTR (CFTR amplifiers) and compounds that increase the plasma membrane stability of mutated CFTR (CFTR stabilizers. CFTR modulators can also enhance function of non-mutated (i.e. wildtype) CFTR and are therefore being studied in disorders where increasing wildtype CFTR function would have beneficial effects in non-CF disorders such as chronic bronchitis/COPD/bronchiectasis (Le Grand, J Med Chem. 2021, 64(11):7241-7260. Patel, Eur Respir Rev. 2020, 29(156):190068) and dry eye disease (Flores, FASEB J. 2016, 30(5):1789-1797).

CFTR modulators and their combinations can be discovered and optimized by assessing their ability to promote trafficking and function of mutated CFTR in in vitro cultivated recombinant and primary cellular systems. Activity in such systems is predictive of activity in CF patients.

The present crystalline CFTR modulator may be useful, alone, or in combination, for the treatment of CFTR-related diseases and disorders, especially cystic fibrosis, or of other CFTR-related diseases and disorders selected from:
chronic bronchitis; rhinosinusitis; constipation; pancreatitis; pancreatic insufficiency; male infertility caused by congenital bilateral absence of the vas deferens (CBAVD); mild pulmonary disease; allergic bronchopulmonary aspergillosis (ABPA); liver disease; coagulation-fibrinolysis deficiencies, such as protein C deficiency; and diabetes mellitus;
asthma; COPD; smoke induced COPD; and dry-eye disease; and
idiopathic pancreatitis; hereditary emphysema; hereditary hemochromatosis; lysosomal storage diseases such as especially I-cell disease pseudo-Hurler; mucopolysaccharidoses; Sandhoff/Tay-Sachs; osteogenesis imperfecta; Fabry disease; Sjogren's disease; osteoporosis; osteopenia; bone healing and bone growth (including bone repair, bone regeneration, reducing bone resorption and increasing bone deposition); chloride channelopathies, such as myotonia congenita (Thomson and Becker forms); Bartter's syndrome type 3; epilepsy; lysosomal storage disease; Primary Ciliary Dyskinesia (PCD)—a term for inherited disorders of the structure and or function of cilia (including PCD with situs inversus also known as Kartagener syndrome, PCD without situs inversus, and ciliary aplasia); generalized epilepsy with fibrile seizures plus (GEFS+); general epilepsy with febrile and afebrile seizures; myotonia; paramyotonia congenital; potassiumaggravated myotonia; hyperkalemic periodic paralysis; long QT syndrome (LQTS); LQTS/Brugada syndrome; autosomal-dominant LQTS with deafness; autosomal-recessive LQTS; LQTS with dysmorphic features; congenital and acquired LQTS; dilated cardiomyopathy; autosomal-dominant LQTS; osteopetrosis; and Bartter syndrome type 3.

WO2019/161078 discloses macrocycles as modulators of cystic fibrosis, wherein said macrocycles generally are 15-membered macrocycles comprising a (pyridine-carbonyl)-sulfamoyl moiety that is linked to a further aromatic group. Further macrocycles are disclosed in WO2022/109573 (macrocycles containing a 1,3,4-oxadiazole ring), WO2022/076625, WO2022/076626, WO2022/076624, WO2022/076621, WO2022/076620, WO2022/076618, WO2021/030556, and WO2021/030555. Macrocyclic tetrapeptides (12- or 13-membered) including the compound Apicidin (CAS: 183506-66-3) have been proposed as potential agents for treating CF (Hutt D M et al. ACS Med Chem Lett. 2011; 2(9):703-707. doi:10.1021/ml200136e). WO2020/128925 discloses macrocycles capable of modulating the activity of CFTR, wherein said macrocycles comprise an optionally substituted divalent N-(pyridine-2-yl)pyridinyl-sulfonamide moiety. Other macrocyclic compounds have been described to stabilize chloride channel CFTR (Stevers L. M., Nature Communications 2022, 13:3586). Non macrocyclic CFTR correctors and/or potentiators of CFTR have been disclosed for example in WO2011/119984, WO2014/015841, WO2007/134279, WO2010/019239, WO2011/019413, WO2012/027731, WO2013/130669, WO2014/078842 and WO2018/227049, WO2010/037066, WO2011/127241, WO2013/112804, WO2014/071122, and WO2020/128768. Furthermore, particular macrocycles can be found as screening compounds (CAS registry number: CAS-2213100-89-9, CAS-2213100-96-8, CAS-2213100-99-1, CAS-2213101-02-9, CAS-2213101-04-1, CAS-2213101-06-3, CAS-2213101-08-5, CAS-2213101-09-6, CAS-2213101-19-8, CAS-2213101-24-5, CAS-2215788-95-5, CAS-2215788-98-8, CAS-2215789-01-6, CAS-2215789-02-7, CAS-2215789-09-4, CAS-2215789-15-2, CAS-2215789-20-9, CAS-2215789-24-3, CAS-2215789-35-6, CAS-2215789-37-8, CAS-2215946-94-2, CAS-2215947-04-7, CAS-2215947-13-8, CAS-2215947-24-1, CAS-2215947-34-3, CAS-2215947-44-5, CAS-2215947-51-4, CAS-2215947-64-9, CAS-2215947-68-3, CAS-2215947-78-5, CAS-2215947-91-2, CAS-2215954-57-5, CAS-2216342-34-4, CAS-2216342-78-6, CAS-2216342-86-6, CAS-2216343-03-0, CAS-2216343-09-6, CAS-2216343-14-3, CAS-2216343-18-7, CAS-2216343-24-5, CAS-2216343-32-5, CAS-2216343-38-1, CAS-2216343-45-0, CAS-2216343-53-0, CAS-2216343-59-6, CAS-2216343-64-3, CAS-2216343-74-5, CAS-2216343-76-7).

The present invention provides a novel crystalline form of (3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide which is CFTR modulator, and is useful for the prevention or treatment of diseases which respond to the activation of CFTR, especially cystic fibrosis. In the treatment of cystic fibrosis said crystalline form may also be used in combination with one or more CFTR modulators known in the art.

### DESCRIPTION OF THE FIGURES

FIG. 1 shows the X-ray powder diffraction diagram of COMPOUND in the crystalline form 1 as obtained in Example 8. The X-ray diffractogram measured with method 1 shows characteristic peaks at the following angles in 2θ (relative peak intensitites given in parenthesis): 5.1° (100%), 8.0° (29%), 10.3° (18%), 12.4° (16%), 18.3° (24%), 18.8° (39%), 19.0° (21%), 19.6° (21%), 21.2° (23%), 22.0° (25%).

FIG. 2 shows the TGA curve of the crystalline form 1 of COMPOUND as obtained in Example 8.

FIG. 3 shows the DSC curve of the crystalline form 1 of COMPOUND as obtained in Example 8

FIG. 4 shows the water sorption isotherm of the sample of crystalline form 1 of COMPOUND from Example 6.

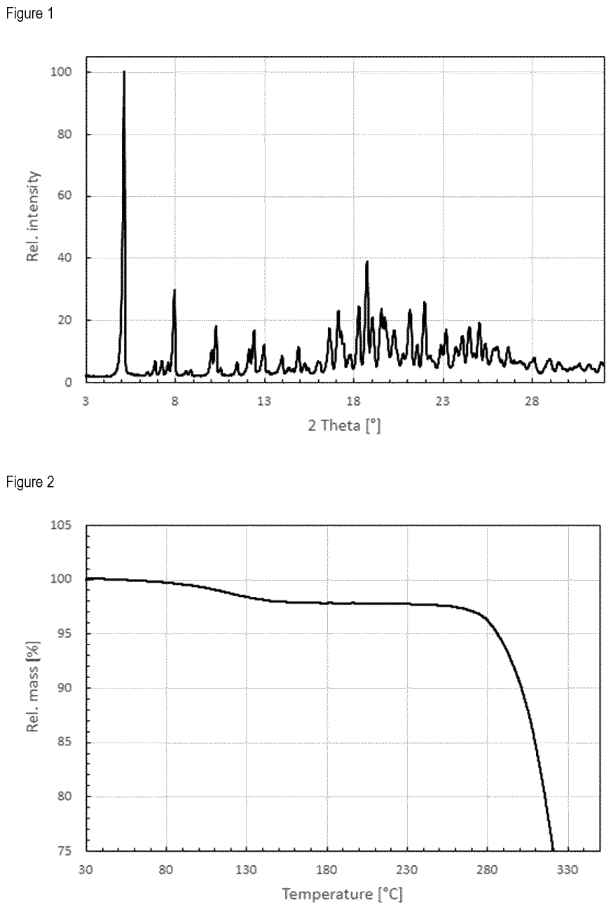

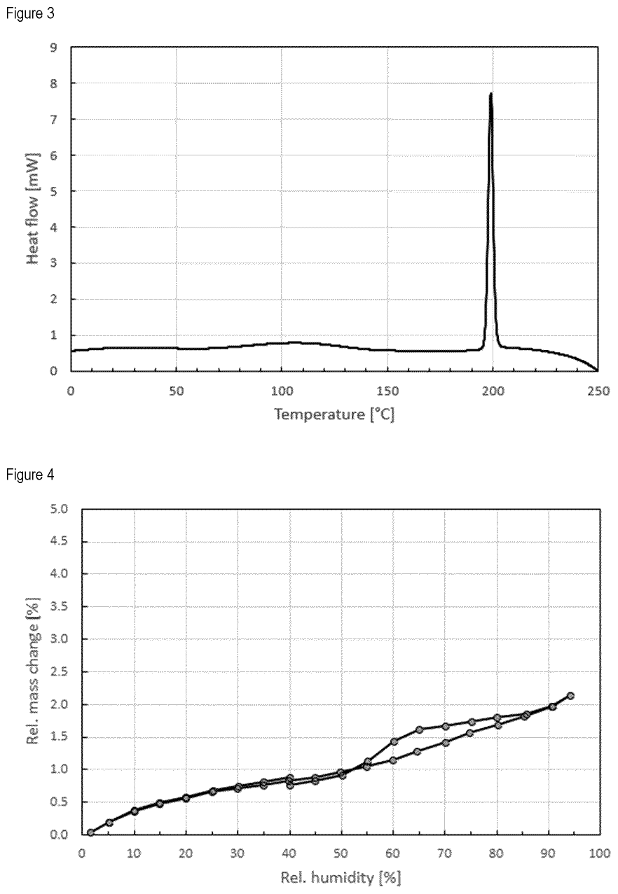

For avoidance of any doubt, the above-listed peaks describe the experimental results of the X-ray powder diffraction shown in FIG. 1. It is understood that, in contrast to the above peak list, only a selection of characteristic peaks is required to fully and unambiguously characterize the COMPOUND in the respective crystalline form of the present invention.

In the X-ray diffraction diagram of FIG. 1 the angle of refraction 2theta (2θ) is plotted on the horizontal axis and the counts on the vertical axis.

### DETAILED DESCRIPTION OF THE INVENTION

1) A first embodiment of the invention relates to a crystalline form of COMPOUND ((3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide

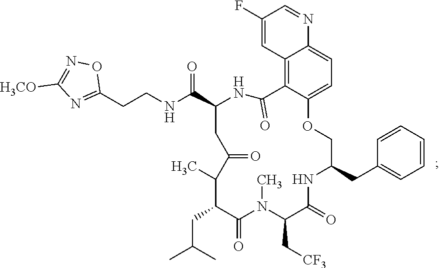 [SMILES: COc1noc(CCNC(=O)[C@@H]2CC(=O)C(C)[C@@H](CC(C)C)C(=O)N(C)[C@H](CC(F)(F)F)C(=O)N[C@H](Cc3ccccc3)COc3ccc4ncc(F)cc4c3C(=O)N2)n1]

characterized by the presence of at least four, or at least six, or at least eight peaks in the X-ray powder diffraction diagram at angles of refraction 2θ selected from: 5.1°, 8.0°, 10.3°, 12.4°, 18.3°, 18.8°, 19.0°, 19.6°, 21.2°, 22.0°.

It is understood that the crystalline form of COMPOUND according to embodiment 1) comprises COMPOUND in crystalline form of the free base (i.e. not in form of a salt). Said crystalline form may comprise non-coordinated and/or coordinated solvent. Coordinated solvent is used herein as term for a crystalline solvate. Likewise, non-coordinated solvent is used herein as term for physisorbed or physically entrapped solvent (definitions according to Polymorphism in the Pharmaceutical Industry (Ed. R. Hilfiker, VCH, 2006), Chapter 8: U. J. Griesser: The Importance of Solvates). Crystalline form of COMPOUND may in particular encompass an isomorphic, non-stoichiometric hydrate, i.e. it may comprise 0 to 1 equivalents of coordinated water. Crystalline form of COMPOUND may in particular encompass isomorphic solvates, i.e. it may comprise coordinated solvent such as isopropanol, methanol, ethanol and/or water.

2) Another embodiment relates to a crystalline form of COMPOUND according to embodiment 1), characterized by the presence of peaks in the X-ray powder diffraction diagram at the following angles of refraction 2θ: 5.1°, 8.0°, 18.8°.

3) Another embodiment relates to a crystalline form of COMPOUND according to embodiments 1) or 2), characterized by the presence of peaks in the X-ray powder diffraction diagram at the following angles of refraction 2θ: 5.1°, 8.0°, 10.3°, 18.8°, 19.6°.

4) Another embodiment relates to a crystalline form of COMPOUND according to any one of embodiments 1) to 3), characterized by the presence of peaks in the X-ray powder diffraction diagram at the following angles of refraction 2θ: 5.1°, 8.0°, 10.3°, 12.4°, 18.3°, 18.8°, 19.0°, 19.6°, 21.2°, 22.0°.

5) Another embodiment relates to a crystalline form of COMPOUND according to any one of embodiments 1) to 4) which essentially shows the X-ray powder diffraction pattern as depicted in FIG. 1.

6) Another embodiment relates to a crystalline form of COMPOUND characterized by the presence of peaks in the X-ray powder diffraction diagram at the following angles of refraction 2θ: 5.1°, 8.0°, 18.8° according to embodiment 1); or to such crystalline form according to any one of embodiments 1) to 5), which exhibits an endothermal event at about 198° C. as determined by differential scanning calorimetry (e.g. by using the method as described herein).

For avoidance of any doubt, whenever one of the above embodiments refers to “peaks in the X-ray powder diffraction diagram at the following angles of refraction 2θ”, said X-ray powder diffraction diagram is obtained by using combined Cu Kα1 and Kα2 radiation, without Kα2 stripping; and it should be understood that the accuracy of the 2θ values as provided herein is in the range of +/−0.1-0.2°. Notably, when specifying an angle of refraction 2theta (2θ) for a peak in the invention embodiments and the claims, the 2θ value given is to be understood as an interval from said value minus 0.2° to said value plus 0.2° (2θ+/−0.2°); and preferably from said value minus 0.1° to said value plus 0.1° (2θ+/−0.1°).

Accordingly, structures are called isomorphic when peak positions in the X-ray powder diffractograms differ not more than by +/−0.2° in 2θ

Where the plural form is used for compounds, solid, pharmaceutical compositions, diseases and the like, this is intended to mean also a single compound, solid, or the like.

The term “enantiomerically enriched” is understood in the context of the present invention to mean especially that at least 90, preferably at least 95, and most preferably at least 99 percent by weight of the COMPOUND are present in form of one enantiomer of the COMPOUND. It is understood that COMPOUND is present in enantiomerically enriched absolute (3S,7S,10R, 13R)-configuration.

The term “essentially pure” is understood in the context of the present invention to mean especially that at least 90, preferably at least 95, and most preferably at least 99 percent by weight of the crystals of a COMPOUND are present in a crystalline form according to the present invention, especially in a single crystalline form of the present invention.

When defining the presence of peak in e.g. an X-ray powder diffraction diagram, a common approach is to do this in terms of the S/N ratio (S=signal, N=noise). According to this definition, when stating that a peak has to be present in an X-ray powder diffraction diagram, it is understood that the peak in the X-ray powder diffraction diagram is defined by having an S/N ratio (S=signal, N=noise) of greater than x (x being a numerical value greater than 1), usually greater than 2, especially greater than 3.

In the context with stating that the crystalline form essentially shows an X-ray powder diffraction pattern as depicted in FIG. 1, the term “essentially” means that at least the major peaks of the diagram depicted in said figures, i.e. those having a relative intensity of more than 10%, especially more than 20%, as compared to the most intense peak in the diagram, have to be present. However, the person skilled in the art of X-ray powder diffraction will recognize that relative intensities in X-ray powder diffraction diagrams may be subject to strong intensity variations due to preferred orientation effects.

Unless used regarding temperatures, the term “about” placed before a numerical value “X” refers in the current application to an interval extending from X minus 10% of X to X plus 10% of X, and preferably to an interval extending from X minus 5% of X to X plus 5% of X. In the particular case of temperatures, the term “about” placed before a temperature “Y” refers in the current application to an interval extending from the temperature Y minus 10° C. to Y plus 10° C., preferably to an interval extending from Y minus 5° C. to Y plus 5° C., notably to an interval extending from Y minus 3° C. to Y plus 3° C. Room temperature means a temperature of about 25° C. When in the current application the term n equivalent(s) is used wherein n is a number, it is meant and within the scope of the current application that n is referring to about the number n, preferably n is referring to the exact number n.

Whenever the word “between” or “to” is used to describe a numerical range, it is to be understood that the end points of the indicated range are explicitly included in the range. For example: if a temperature range is described to be between 40° C. and 80° C. (or 40° C. to 80° C.), this means that the end points 40° C. and 80° C. are included in the range; or if a variable is defined as being an integer between 1 and 4 (or 1 to 4), this means that the variable is the integer 1, 2, 3, or 4.

The expression % w/w refers to a percentage by weight compared to the total weight of the composition considered. Likewise, the expression v/v refers to a ratio by volume of the two components considered. The expression “vol” signifies volumes (in L, e.g. of solvent) per weight (in kg, e.g. of reactant). For example 7 vol signifies 7 liters (of solvent) per kg (of reactant).

The crystalline form, especially the essentially pure crystalline form, of COMPOUND according to any one of embodiments 1) to 6) can be used as medicament, e.g. in the form of pharmaceutical compositions for enteral or parenteral administration.

7) Another embodiment thus relates to a crystalline form of COMPOUND (3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide according to any one of embodiments 1) to 6) for use as a medicament.

The crystalline solid, especially the essentially pure crystalline solid, of COMPOUND according to any one of embodiments 1) to 6) may be used as single component or as mixtures with other crystalline forms or the amorphous form of COMPOUND.

The production of the pharmaceutical compositions can be effected in a manner which will be familiar to any person skilled in the art (see for example Remington, The Science and Practice of Pharmacy, 21st Edition (2005), Part 5, “Pharmaceutical Manufacturing” [published by Lippincott Williams & Wilkins]) by bringing the crystalline form of the present invention, optionally in combination with other therapeutically valuable substances, into a galenical administration form together with suitable, non-toxic, inert, pharmaceutically acceptable solid or liquid carrier materials and, if desired, usual pharmaceutical adjuvants.

8) A further embodiment of the invention relates to pharmaceutical compositions comprising as active ingredient a crystalline form of COMPOUND (3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide according to any one of embodiments 1) to 6), and at least one pharmaceutically acceptable carrier material.

Such pharmaceutical compositions according to embodiment 8) are especially useful for the prevention or treatment of CFTR-related diseases or disorders, especially of cystic fibrosis.

9) A further embodiment of the invention relates to a pharmaceutical composition according to embodiment 8), wherein said pharmaceutical composition is in form of a tablet.

10) A further embodiment of the invention relates to a pharmaceutical composition according to embodiment 8), wherein said pharmaceutical composition is in form of a capsule.

11) A further embodiment of the invention relates to a pharmaceutical composition according to embodiment 8), wherein said pharmaceutical composition is in liquid form.

12) A further embodiment of the invention relates to the use of a crystalline form of COMPOUND (3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide according to any one of embodiments 1) to 6), in the manufacture of a pharmaceutical composition, wherein said pharmaceutical composition comprises as active ingredient the COMPOUND (3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide, and at least one pharmaceutically acceptable carrier material.

For avoidance of any doubt, embodiment 12) refers to the crystalline form according to any one of embodiments 1) to 6) which is suitable/which is used as final isolation step of COMPOUND (e.g. in order to meet the purity requirements of pharmaceutical production), whereas the final pharmaceutical composition according to embodiment 12) may or may not contain said crystalline form (e.g. because the originally crystalline form of COMPOUND is further transformed during the manufacturing process and/or is dissolved in the pharmaceutically acceptable carrier material(s); thus, in the final pharmaceutical composition, COMPOUND may be present in non-crystalline form, in another crystalline form, or in dissolved form, or the like).

13) A further embodiment of the invention thus relates to a pharmaceutical composition comprising as active ingredient the COMPOUND (3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide, wherein said pharmaceutical composition is manufactured using a crystalline form of COMPOUND (3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide according to any one of embodiments 1) to 6) and at least one pharmaceutically acceptable carrier material.

14) A further embodiment of the invention relates to a pharmaceutical composition according to embodiment 13), wherein said pharmaceutical composition is in form of a capsule, tablet, or in liquid form.

15) A further embodiment of the invention relates to a crystalline form of COMPOUND ((3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide according to any one of embodiments 1) to 6), for use in the prevention/prophylaxis or treatment of CFTR-related diseases or disorders, especially cystic fibrosis.

16) A further embodiment of the invention relates to a crystalline form of COMPOUND (3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide according to any one of embodiments 1) to 6), for use in the preparation of a medicament for the prevention/prophylaxis or treatment of CFTR-related diseases or disorders, especially cystic fibrosis.

17) A further embodiment of the invention relates to a method of treatment of CFTR-related diseases, especially of cystic fibrosis, comprising administering to a patient an effective amount of a crystalline form of the compound ((3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide according to any one of embodiments 1) to 6).

The crystalline form of COMPOUND as defined in any one of embodiments 1) to 6) are useful for the treatment of CFTR-related diseases or disorders, especially cystic fibrosis.

CFTR-related diseases and disorders may be defined as including especially cystic fibrosis, as well as further CFTR-related diseases and disorders selected from:
chronic bronchitis; rhinosinusitis; constipation; pancreatitis; pancreatic insufficiency; male infertility caused by congenital bilateral absence of the vas deferens (CBAVD); mild pulmonary disease; allergic bronchopulmonary aspergillosis (ABPA); liver disease; coagulation-fibrinolysis deficiencies, such as protein C deficiency; and diabetes mellitus;
asthma; COPD; smoke induced COPD; and dry-eye disease; and
idiopathic pancreatitis; hereditary emphysema; hereditary hemochromatosis; lysosomal storage diseases such as especially I-cell disease pseudo-Hurler; mucopolysaccharidoses; Sandhoff/Tay-Sachs; osteogenesis imperfecta; Fabry disease; Sjogren's disease; osteoporosis; osteopenia; bone healing and bone growth (including bone repair, bone regeneration, reducing bone resorption and increasing bone deposition); chloride channelopathies, such as myotonia congenita (Thomson and Becker forms); Bartter's syndrome type 3; epilepsy; lysosomal storage disease; Primary Ciliary Dyskinesia (PCD)—a term for inherited disorders of the structure and or function of cilia (including PCD with situs inversus also known as Kartagener syndrome, PCD without situs inversus, and ciliary aplasia); generalized epilepsy with fibrile seizures plus (GEFS+); general epilepsy with febrile and afebrile seizures; myotonia; paramyotonia congenital; potassium-aggravated myotonia; hyperkalemic periodic paralysis; long QT syndrome (LQTS); LQTS/Brugada syndrome; autosomal-dominant LQTS with deafness; autosomal-recessive LQTS; LQTS with dysmorphic features; congenital and acquired LQTS; dilated cardiomyopathy; autosomal-dominant LQTS; osteopetrosis; and Bartter syndrome type 3.

The term “treatment of cystic fibrosis” refers to any treatment of cystic fibrosis and includes especially treatment that reduces the severity of cystic fibrosis and/or reduces the symptoms of cystic fibrosis.

The term “cystic fibrosis” refers to any form of cystic fibrosis, especially to a cystic fibrosis that is associated with one or more gene mutation(s). Preferably, such cystic fibrosis is associated with an CFTR trafficking defect (class II mutations) or reduced CFTR stability (class VI mutations) [in particular, an CFTR trafficking defect/class II mutation], wherein it is understood that such CFTR trafficking defect or reduced CFTR stability may be associated with another disease causing mutation of the same or any other class. Such further disease causing CFTR gene mutation comprises class I mutations (no functional CFTR protein), (a further) class II mutation (CFTR trafficking defect), class III mutations (CFTR regulation defect), class IV mutations (CFTR conductance defect), class V mutations (less CFTR protein due to splicing defects), and/or (a further) class VI mutation (less CFTR protein due to reduced CFTR stability). Said one or more gene mutation(s) may for example comprise at least one mutation selected from F508del, A561E, and N1303K, as well as 1507del, R560T, R1066C and V520F; in particular F508del. In addition to the above-listed, further CFTR gene mutations comprise for example G85E, R347P, L206W, and M1101K. Said gene mutation(s) may be heterozygous, homozygous or compound hetereozygous. Especially said gene mutation is heterozygous comprising one F508del mutation. Further CFTR gene mutations (which are especially class III and/or IV mutations) comprise G551D, R117H, D1152H, A455E, S549N, R347H, S945L, and R117C.

The severity of cystic fibrosis/of a certain gene mutation associated with cystic fibrosis as well as the efficacy of correction thereof may generally be measured by testing the chloride transport effected by the CFTR. In patients, for example average sweat chloride content may be used for such assessment.

The term “symptoms of cystic fibrosis” refers especially to elevated chloride concentration in the sweat; symptoms of cystic fibrosis further comprise chronic bronchitis; rhinosinusitis; constipation; pancreatitis; pancreatic insufficiency; male infertility caused by congenital bilateral absence of the vas deferens (CBAVD); mild pulmonary disease; allergic bronchopulmonary aspergillosis (ABPA); liver disease; coagulation-fibrinolysis deficiencies such as protein C deficiency; and/or diabetes mellitus.

The crystalline form of COMPOUND as defined in any one of embodiments 1) to 6) may in particular be useful as therapeutic agents for the prevention/prophylaxis or treatment of a CFTR-related diseases and disorders, especially cystic fibrosis. It can be used as single therapeutic agent or in combination with one or more therapeutically active ingredients acting as CFTR modulator(s), wherein said one or more CFTR modulator(s) is/are CFTR corrector(s), and/or a CFTR potentiator. Such combined treatment may be effected simultaneously, in a fixed dose or in a non-fixed dose.

The invention, thus, also relates to pharmaceutical compositions comprising a pharmaceutically acceptable carrier material, and:
a crystalline form of COMPOUND as defined in any one of embodiments 1) to 6);
and one or more therapeutically active ingredients acting as CFTR modulator(s), wherein said CFTR modulator(s) is/are CFTR corrector(s), and/or a CFTR potentiator.

The term “therapeutically active ingredients acting as CFTR modulator” refers to any CFTR corrector (especially type-I-, type-II-, or type-Ill corrector) and CFTR potentiator that has shown—alone and/or in combination—potential for therapeutic use (as tested in in vitro and/or in vivo models, especially in clinical trials) and/or is indicated for such therapeutic use; wherein such therapeutic use is for CFTR-related disease (in particular cystic fibrosis). Examples are especially CFTR potentiators: ivacaftor, navocaftor, icenticaftor, deutivacaftor, GLPG-1837, and GLPG-2451; and CFTR correctors: type-I correctors (lumacaftor, tezacaftor, galicaftor), type-II correctors (Corrector4a), and type-Ill correctors (elexacaftor, bamocaftor, olacaftor, vanzacaftor).

Preferred is a pharmaceutical composition comprising the crystalline form of COMPOUND as defined in any one of embodiments 1) to 6), wherein said composition further comprises navocaftor and galicaftor. Another preferred pharmaceutical composition comprises the crystalline form of COMPOUND as defined in any one of embodiments 1) to 6), ivacaftor, and tezacaftor.

“Simultaneously”, when referring to an administration type, means in the present application that the administration type concerned consists in the administration of two or more active ingredients and/or treatments at approximately the same time; wherein it is understood that a simultaneous administration will lead to exposure of the subject to the two or more active ingredients and/or treatments at the same time. When administered simultaneously, said two or more active ingredients may be administered in a fixed dose combination, or in an equivalent non-fixed dose combination (e.g. by using two or more different pharmaceutical compositions to be administered by the same route of administration at approximately the same time), or by a non-fixed dose combination using two or more different routes of administration; wherein said administration leads to essentially simultaneous exposure of the subject to the two or more active ingredients and/or treatments.

“Fixed dose combination”, when referring to an administration type, means in the present application that the administration type concerned consists in the administration of one single pharmaceutical composition comprising the two or more active ingredients.

The present invention also relates to a process for the preparation of COMPOUND in enantiomerically enriched form, and to processes for the preparation and characterization of the crystalline form of COMPOUND according to any one of embodiments 1) to 6). Said processes are described in the procedures of the experimental part below.

### EXPERIMENTAL PROCEDURES

All temperatures are stated in ° C. Commercially available starting materials are used as received without further purification. Unless otherwise specified, all reactions are carried out in oven-dried glassware under an atmosphere of nitrogen or argon. Compounds are purified by flash column chromatography on silica gel or by preparative HPLC. Compounds described in the invention are characterised by LC-MS data (retention time t_{R} is given in min; molecular weight obtained from the mass spectrum is given in g/mol) using the conditions listed below. In cases where compounds of the present invention appear as a mixture of conformational isomers, particularly visible in their LC-MS spectra, the retention time of the most abundant conformer is given.

### Quality Control (QC) Analytical LC-MS:

### Equipment and Conditions:

Pump: Waters Acquity Binary, Solvent Manager, MS: Waters SQ Detector, DAD: Acquity UPLC PDA Detector, ELSD: Acquity UPLC ELSD. Columns: Acquity UPLC CSH C18 1.7 μm 2.1×50 mm or Acquity UPLC HSS T3 C18 1.8 μm 2.1×50 mm from Waters, thermostated in the Acquity UPLC Column Manager at 60° C. Eluents: A1: H₂O+0.05% FA; B1: AcCN+0.045% FA. Method: Gradient: 2% B 98% B over 2.0 min. Flow: 1.0 mL/min. Detection: UV 214 nm and ELSD, and MS, tR is given in min.

### Analytical LC-MS

### Equipment:

Binary gradient pump Agilent G4220A or equivalent with mass spectrometry detection (single quadrupole mass analyser, Thermo Finnigan MSQPIus or equivalent).

### Conditions:

Method B (acidic): Column: Zorbax RRHD SB-aq (1.8 μm, 2.1×50 mm). Conditions: MeCN [eluent A]; water+0.04% TFA [eluent B]. Gradient: 95% B→5% B over 2.0 min (flow: 0.8 mL/min). Detection: UV/Vis+MS.
Method I (basic): Column: Waters BEH C18 (2.5 μm, 2.1×50 mm). Conditions: water/NH₃ [c(NH₃)=13 mmol/l][eluent A]; MeCN [eluent B]. Gradient: 5% B→95% B over 2 min (flow 0.8 mL/min). Detection: UVNis+MS.
Method J (basic): Column: Waters XSelect CSH C18 (3.5 μm, 2.1×30 mm). Conditions: 95% MeCN+5% Water/NH₄HCO₃ [c(NH₄HCO₃)=10 mmol/l][eluent A]; Water/NH₄HCO₃ [c(NH₄HCO₃)=10 mmol/l][eluent B]. Gradient: 95% B→2% B over 1.6 min (flow 1 mL/min), Detection: UV/Vis+MS.

### Preparative LC-MS

### Equipment:

Binary gradient pump Gilson 333/334 or equivalent with mass spectrometry detection (single quadrupole mass analyser, Thermo Finnigan MSQPIus or equivalent).

### Conditions:

Basic conditions: Column: Waters XBridge C18 (10 μm, 30×75 mm); conditions: MeCN [eluent A]; water+0.5% NH₄OH (25% aq.) [eluent B]; gradient: 95% B→5% B, over 6.5 min (flow: 75 mL/min). Detection: UV/Vis+MS.

### Chiral Analytical Chromatography

### Equipment:

HPLC: Dionex HPG-3200SD pump with a Dionex DAD-3000 UV detector.

SFC: CO₂ supply: Aurora Fusion A5 Evolution; pump: Agilent G4302A; UV detector: Agilent G1315C.

### Conditions:

HPLC: Columns: ChiralPak AY-H, 5 μm, 250×4.6 mm or Regis (R,R) Whelk-O1 250×4.6 mm, 5 μm; eluent: A: Hept, 0.05% DEA, B: Ethanol, 0.05% DEA, flow 0.8 to 1.2 mL/min.
SFC Column: Regis (R,R) Whelk-O1, 4.6×250 mm, 5 μM; eluent: A: 60% CO₂, B: 40% DCM/EtOH/DEA 50:50:0.1

### Chiral Preparative Chromatography

### Equipment:

HPLC: 2 Varian SD1 Pump with a Dionex DAD-3000 UV Detector.
SFC: CO₂ supply: Maximator DLE15-GG-C; pumps: 2 SSI HF CP 300; UV detector: Dionex DAD-3000.

### Conditions:

HPLC: Columns: ChiralPak IA, IB, IC, IE, or IF, 5 μm, 20×250 mm, or Regis (R,R) Whelk-O1, 21.1×250 mm, 5 μm; eluent: appropriate mixture of A (0% to 90% Hept) and B (10% to 100% EtOH, 0.1% DEA), flow: appropriate flow of 16, 23 or 34 mL/min.
SFC: Columns: Regis (R,R) Whelk-O1, 30×250 mm, 5 μm or ChiralPak IC, 30×250 mm, 5 μm; eluent: appropriate mixture of A (60% to 80% CO₂), and B (30% to 40% of DCM/EtOH/DEA 50:50:0.1), flow 160 mL/min.

### X-Ray Powder Diffraction Analysis (XRPD)

### XRPD (Method 1):

X-ray powder diffraction patterns are collected on a Bruker D8 Advance X-ray diffractometer with a CuKαLX-ray tube, which is run at 40 kV/40 mA, and a Lynxeye linear detector. The instrument is operated in reflection mode (coupled two Theta/Theta) with a step size of 0.02° (2θ) and a step time of 76.8 sec over a scanning range from 3° to 50° in 2θ. The divergence slit is set to variable slit opening for full sample irradiation at all angles, and the antiscatter slit on the detector side is opened at maximum. The powder is filled into the cavity of a silicon single crystal sample holder with a diameter of 25 mm and a depth of 0.5 mm and evened out with a glass slide. The samples are rotated in their own plane during the measurement. Diffraction data are reported using combined Cu Kα1 and Kα2 radiation, without Kα2 stripping. The accuracy of the 2θ values as provided herein is in the range of +/−0.2° as it is generally the case for conventionally recorded X-ray powder diffraction patterns.

### XRPD (Method 2):

X-ray powder diffraction patterns are collected on a Bruker D8 GADDS-HTS diffractometer equipped with an automated XYZ stage, laser video microscope for sample positioning, a Vantec-500 detector and a CuKα-X-ray tube, which is run at 40 kV/40 mA. The instrument is operated in the reflection mode, and the X-ray optics consists of a single Göbel multilayer mirror coupled with a pinhole collimator of 0.5 mm. Typically a single frame is collected over 180 s with goniometer positions of theta1 at 4° and theta2 at 16° and a sample-detector distance of 20 cm. The frame is integrated in the range of 5-35° 2θ. Samples are measured under ambient conditions and are prepared as flat plate specimens using powder as received without grinding. Approximately 5-10 mg of sample is lightly pressed on a glass slide to obtain a flat surface. The sample is not moved during the measurement. Diffraction data are reported using combined Cu Kα1 and Kα2 radiation, without Kα2 stripping. The accuracy of the 20 values as provided herein is in the range of +/−0.2° as it is generally the case for conventionally recorded X-ray powder diffraction patterns.

### Gravimetric Vapour Sorption (GVS) Analysis

Measurements are performed on a multi-sample instrument SPS-100n (Projekt Messtechnik, Ulm, Germany), which is operated in stepping mode at 25° C. A sample of approximately 20 mg is equilibrated at 40% RH before starting a pre-defined humidity program of 40-0-95-0-95-40% RH with steps of 5% ARH and a maximum equilibration time of 24 h per step. The hygroscopic classification is done according to the European Pharmacopeia Technical Guide (1999, page 86), e.g., non-hygroscopic: increase in mass less than 0.2% mass/mass; slightly hygroscopic: increase in mass is less than 2% and equal to or greater than 0.2% mass/mass; hygroscopic: increase in mass is less than 15% and equal to or greater than 2% mass/mass. The mass change between 40% relative humidity and 80% relative humidity in the first adsorption scan is evaluated for this classification.

### Differential Scanning Calorimetry (DSC)

DSC data are collected on a Mettler Toledo DSC 3⁺ STAR^{e} system with STAR^{e} software version 16.00. The instrument is calibrated for energy and temperature using certified indium. Typically, 1-5 mg of sample are placed in an automatically pierced aluminum pan, it is then heated from −20° C. to 250° C. at a rate of 10° C. min⁻¹. A nitrogen purge at 20 mL min⁻¹ is maintained over the sample. Peak temperatures are reported for melting points.

Thermogravimetric analysis (TGA) TGA data are collected on a Mettler Toledo TGA/DSC 3⁺ STAR^{e} system. Typically, about 5 mg of a sample are placed in an automatically pierced aluminum pan and heated at 10° C. min⁻¹ from 30° C. to 350° C. under a constant flow of nitrogen. The off-gases are analyzed with a Pfeiffer ThermoStar quadrupole mass spectrometer.

### ABBREVIATIONS(as Used Hereinbefore or Hereinafter)

aq. aqueous
Boc butyloxycarbonyl
d day(s)
DCM dichloromethane
DEA diethylamine
DIPEA diisopropyl-ethylamine, Hunig's base, ethyl-diisopropylamine
DMF dimethylformamide
DMSO dimethylsulfoxide
Et ethyl
EtOAc ethyl acetate
EtOH ethanol
FC flash chromatography
h hour(s)
HATU 2-(7-Aza-1H-benzotriazole-1-yl)-1,1,3,3-tetramethyluronium hexafluorophosphate
Hept heptane
HPLC high performance liquid chromatography
HV high vacuum conditions
LC-MS liquid chromatography—mass spectrometry
Me methyl
MeCN acetonitrile
MeOH methanol
mL milliliter
min minute(s)
m.p. melting peak
Ph phenyl
prep. Preparative
rpm rounds per minute
RT room temperature
RH relative humidity
s second(s)
sat. Saturated
SEM Scanning Electron Microscopy
SFC supercritical fluid chromatography
tBME tert.-butyl methyl ether
tBu tert-butyl=tertiary butyl
TFA trifluoroacetic acid
TH F tetrahydrofuran
tR retention time

### Synthesis of Reference Example 1

### Building Block Synthesis

### tert-Butyl (S)-3-amino-4-((2-(3-methoxy-1,2,4-oxadiazol-5-yl)ethyl)amino)-4-oxobutanoate (A-1)

Step 1: HATU (11.82 g, 31.1 mmol) is added to a RT soln. of boc-beta-Ala-OH (5.0 g, 25.9 mmol), o-methylisourea bisulfate (4.5 g, 25.9 mmol, and DIPEA (18.1 mL, 104 mmol) in DMF (150 mL) and the RM is stirred for 1.5 h. Water and EtOAc are added to the RM, then the two layers are separated and the aq. layer is extracted with EtOAc (2×). The combined org. extracts are washed with brine, dried (Na₂SO₄), filtered, and concentrated to give the crude product that is purified by FC (eluting with 20% to 100% EtOAc in hept) to give tert-butyl (3-((imino(methoxy)methyl)amino)-3-oxopropyl)carbamate as a white solid. LC-MS I: t_{R}=0.64 min; [M+H]⁺=246.36.

Step 2: 1,8-Diazabicyclo[5.4.0]undec-7-ene (8.96 mL, 59.3 mmol) is added to a RT soln. of tert-butyl (3-((imino(methoxy)methyl)amino)-3-oxopropyl)carbamate (6.19 g, 24.7 mmol) and NBS (10.56 g, 59.3 mmol) in EtOAc (120 mL) and the RM is stirred for 5 h. Additional 1,8-diazabicyclo[5.4.0]undec-7-ene (1.85 mL, 12.4 mmol) and NBS (2.2 g, 12.4 mmol) are added and stirring is continued for 16 h. The suspension is filtered and the filtrate is washed with water, sat. aq. NaHCO₃ soln. and brine before being evaporated to dryness. The crude product is purified by FC (eluting with 20% to 100% EtOAc in hept) to give tert-butyl (2-(3-methoxy-1,2,4-oxadiazol-5-yl)ethyl)carbamate as a colourless oil. LC-MS I: t_{R}=0.75 min; [M+H]⁺=244.33.

Step 3: 4 M HCl in dioxane (0.62 mL, 2.47 mmol) is added to a RT soln. of tert-butyl (2-(3-methoxy-1,2,4-oxadiazol-5-yl)ethyl)carbamate (150 mg, 0.62 mmol) in DCM (2 mL) and the RM is stirred for 4 days at RT, then at 50° C. for 6 h. The mixture is evaporated to give 2-(3-methoxy-1,2,4-oxadiazol-5-yl)ethan-1-amine hydrochloride as a white solid. LC-MS I: t_{R}=0.35 min; [M+H]⁺=144.21.

Step 4: HATU (7.36 g, 19.4 mmol) is added to a RT soln. of Fmoc-L-aspartic acid beta-tert-butyl ester (7.74 g, 18.4 mmol), 2-(3-methoxy-1,2,4-oxadiazol-5-yl)ethan-1-amine hydrochloride (3.31 g, 18.4 mmol) and DIPEA (9.47 mL, 55.3 mmol) in DMF (121 mL) and the RM is stirred for 1 h. The RM is partitioned between water and EtOAc and the layers are separated. The aq. phase is re-extracted with EtOAc (2×) and the combined org. extracts are washed with sat. aq. NaHCO₃, brine, dried over Na₂SO₄, filtered and evaporated to give tert-butyl (S)-3-((((9H-fluoren-9-yl)methoxy)carbonyl)amino)-4-((2-(3-methoxy-1,2,4-oxadiazol-5-yl)ethyl)amino)-4-oxobutanoate as a white solid. LC-MS I: t_{R}=1.08 min; [M+H]⁺=537.66.

Step 5: Piperidine (9.38 mL, 93.9 mmol) is added to a RT soln. of tert-butyl (S)-3-((((9H-fluoren-9-yl)methoxy)carbonyl)amino)-4-((2-(3-methoxy-1,2,4-oxadiazol-5-yl)ethyl)amino)-4-oxobutanoate (10.39 g, 18.8 mmol) in DCM (170 mL) and the RM is stirred for 16 h. The RM is concentrated and the residue is triturated at 0° C. with MeCN before being filtered. The filtrate is washed with heptane (2×), concentrated and purified by FC (eluting with 10% MeOH in DCM) to give the title compound A-1 as a white solid. LC-MS I: t_{R}=0.63 min; [M+H]⁺=315.39.

### Benzyl (R)-6-(2-amino-3-phenylpropoxy)-3-fluoroquinoline-5-carboxylate dihydrochloride (B-1)

Step 1: A soln. of Br₂ (0.17 mL, 3.37 mmol) in AcOH (8.0 mL) is added to a RT soln. of 3-fluoroquinolin-6-ol (0.50 g, 3.06 mmol) and NaOAc (0.30 g, 3.68 mmol) in AcOH (20 mL) and the RM is stirred for 30 min. The RM is concentrated to dryness, the residue is partitioned between sat. aq. NaHCO₃ and EtOAc and extracted. The layers are separated, and the aq. layer is re-extracted with EtOAc (2×). The combined org. extracts are washed with brine, dried (Na₂SO₄), filtered, and evaporated to give 5-bromo-3-fluoroquinolin-6-ol as a brown solid. LC-MS J: t_{R}=1.38 min; [M+H]⁺=239.9.

Step 2: A soln. of 5-bromo-3-fluoroquinolin-6-ol (0.74 g, 3.06 mmol) in THE (15 mL) is added dropwise to a RT suspension of NaH (0.17 g, 4.29 mmol) in THE (15 mL) and the resulting mix. is stirred for 15 min before methoxymethyl bromide (0.3 mL, 3.67 mmol) is added dropwise at 0° C. After stirring for 1.5 h at 0° C. the RM is quenched by the addition of H₂O and extracted with EtOAc. The org. layer is washed with NaHCO₃, brine, dried (Na₂SO₄), filtered, and evaporated. The crude product is purified by FC (eluting with 2% to 30% EtOAc in hept) to give 5-bromo-3-fluoro-6-(methoxymethoxy)quinoline as a colourless oil. LC-MS J: t_{R}=2.03 min; No ionisation.

Step 3: nBuLi (1.6 M in hex, 0.98 mL, 1.57 mmol) is added dropwise to a −78° C. soln. of 5-bromo-3-fluoro-6-(methoxymethoxy)quinoline (300 mg, 1.05 mmol) in THE (18 mL) and the RM is stirred for 30 min. The RM is quenched with freshly ground dry ice (1.0 g, 22.7 mmol) and then warmed to RT and stirred for 30 min. The RM is concentrated in vacuo and the intermediate lithium carboxylate is dissolved in DMF (4 mL), then KHCO₃ (31.5 mg, 0.315 mmol) and BnBr (0.15 mL, 1.26 mmol) are added, and the RM is stirred at RT for 16 h. The RM is partitioned between sat. aq. NaHCO₃ and EtOAc and extracted. The layers are separated, and the aq. layer is re-extracted with EtOAc (2×). The combined org. extracts are washed with brine, dried (Na₂SO₄), filtered, and evaporated. The crude product is purified prep. HPLC (basic) to give benzyl 3-fluoro-6-(methoxymethoxy)quinoline-5-carboxylate as a yellow oil. LC-MS J: t_{R}=2.08 min; [M+H]⁺=342.10.

Step 4: TFA (0.24 mL, 3.13 mmol) is added to a RT soln. of benzyl 3-fluoro-6-(methoxymethoxy)quinoline-5-carboxylate (107 mg, 0.31 mmol) in DCM (3 mL) and the resulting mix. is stirred for 2 h. The RM is concentrated in vacuo, the residue dissolved in EtOAc, and extracted with aq. sat. NaHCO₃ soln. The aq. layer is extracted with EtOAc and the combined org. extracts are washed with brine, dried (NaSO₄), filtered, and concentrated to give benzyl 3-fluoro-6-hydroxyquinoline-5-carboxylate as a light brown oil. LC-MS J: t_{R}=2.06 min; [M+H]⁺=298.1.

Step 5: DIAD (0.064 mL, 0.33 mmol) is added to a 0° C. mix. of benzyl 3-fluoro-6-hydroxyquinoline-5-carboxylate (93.8 mg, 0.31 mmol), tert-butyl (R)-(1-hydroxy-3-phenylpropan-2-yl)carbamate (82 mg, 0.33 mmol) and PPh₃ (86 mg, 0.33 mmol) in THE (2 mL), and the RM is stirred for 16 h at RT. The mix. is concentrated and the residue directly purified by FC (eluting with 20% to 60% EtOAc in hept) to give benzyl (R)-6-(2-((tert-butoxycarbonyl)amino)-3-phenylpropoxy)-3-fluoroquinoline-5-carboxylate as a colourless oil. LC-MS J: t_{R}=2.39 min; [M+H]⁺=531.2.

Step 6: 4M HCl in dioxane (0.44 mL, 1.77 mmol) is added to a soln. benzyl (R)-6-(2-((tert-butoxycarbonyl)amino)-3-phenylpropoxy)-3-fluoroquinoline-5-carboxylate (94 mg, 0.18 mmol) in dioxane (3 mL) and the RM is stirred for 24 h at RT. The volatiles are removed in vacuo and the residue is triturated with Et₂O (3×) to give the title compound B-1 as a white solid. LC-MS J: t_{R}=2.10 min; [M+H]⁺=431.2.

### 2-((S)-2-((tert-Butoxycarbonyl)(methyl)amino)-N,4-dimethylpentanamido)-4,4,4-trifluorobutanoic acid (C-1)

Step 1: NaOAc (12.78 g, 0.156 mol), TFA (2.41 mL, 31.1 mmol) and benzaldehyde (3.34 mL, 32.7 mmol) are added to a RT soln. of methyl 2-amino-4,4,4-trifluorobutanoate hydrochloride (6.81 g, 31.1 mmol) in MeOH (20 mL) and the resulting mix. is stirred for 1 h. NaBH₃CN (2.27 g, 34.3 mmol) is then added and stirring is continued for 45 min. The mixture is evaporated to dryness, then partitioned between H₂O and DCM, and the layers are separated. The aq. layer is extracted with DCM and the combined org. extracts are dried (Na₂SO₄), filtered, and evaporated to give methyl-2-(benzylamino)-4,4,4-trifluorobutanoate as a brown oil, which is used as such in the next step. LC-MS I: t_{R}=0.96 min; [M+H]⁺=262.37.

Step 2: NaOAc (12.67 g, 155 mmol), TFA (2.39 mL, 30.9 mmol) and formaldehyde (37% in H₂O, 2.53 mL, 34 mmol) are added to a RT solution of methyl-2-(benzylamino)-4,4,4-trifluorobutanoate (8.07 g, 30.9 mmol) in MeOH (100 mL) and the resulting mix. is stirred at RT for 1 h. NaBH₃CN (2.25 g, 34.0 mmol) is then added and stirring is continued. After 1.5 h, formaldehyde (37% in H₂O, 0.46 mL, 6.18 mmol) and NaBH₃CN (409 mg, 6.18 mmol) are added and the mix. is stirred for another 2 h at RT. The mix. is evaporated to dryness, partitioned between H₂O and DCM, and the layers separated. The aq. layer is re-extracted with DCM and the combined org. extracts are dried (Na₂SO₄), filtered, and evaporated to give methyl-2-(benzyl(methyl)amino)-4,4,4-trifluorobutanoate as a brown oil, which is used as such in the next step. LC-MS I: t_{R}=1.13 min; [M+H]⁺=276.45.

Step 3: A solution of methyl-2-(benzyl(methyl)amino)-4,4,4-trifluorobutanoate (6.48 g, 23.5 mmol) in EtOH (200 mL) is evacuated/purged with Ar (3×) before Pd/C (1.25 g, 5 mol %) is added. The RM is evacuated/purged with H₂ (3×) and stirred under a H₂ atm for 2.5 h. The mix. is filtered and rinsed with MeOH. 4M HCl (5.89 mL, 23.5 mmol) is added and the mix. is evaporated to dryness to give methyl-4,4,4-trifluoro-2-(methylamino)butanoate hydrochloride as an off-white solid which is used as such in the next step. LC-MS I: t_{R}=0.60 min; [M+H]⁺=186.37.

Step 4: HATU (11.26 g, 29.6 mmol) is added portionwise to a RT soln. of Boc-N-methyl-L-leucine (6.24 g, 24.7 mmol), methyl-4,4,4-trifluoro-2-(methylamino)butanoate hydrochloride (5.47 g, 24.7 mmol), and DIPEA (16.9 mL, 98.7 mmol) in DMF (80 mL) and the resulting mix. is stirred for 1 h. Water is added and the mix. is extracted with EtOAc (3×). The combined org. extracts are successively washed with sat. aq. NaHCO₃, H₂O, and brine, dried (Na₂SO₄), filtered, and concentrated. Purification by FC (eluting with 15% EtOAc in hept) gives methyl-2-((S)-2-((tert-butoxycarbonyl)(methyl)amino)-N,4-dimethylpentanamido)-4,4,4-trifluorobutanoate as a yellow oil. LC-MS B: t_{R}=1.06 min; [M+H]⁺=413.29.

Step 5: 2 M aq. NaOH (6.9 mL, 13.8 mmol) is added to a RT soln. of methyl-2-((S)-2-((tert-butoxycarbonyl)(methyl)amino)-N,4-dimethylpentanamido)-4,4,4-trifluorobutanoate (2.84 g, 6.88 mmol) in MeOH (10 mL) and the mix. is stirred at RT for 1.5 h. The volatiles are removed in vacuo and the aq. residue is neutralised with 2 M aq. HCl before being extracted with DCM (3×). The combined org. layers are dried (Na₂SO₄), filtered, and evaporated to give the title compound C-1 as a white solid. LC-MS B: t_{R}=0.96 min; [M+H]⁺=399.29.

### Reference Example 1: (3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide

Step 1: HATU (1.03 g, 2.58 mmol) is added to a RT soln. of B-1 (1.24 g, 2.46 mmol), C-1 (980 mg, 2.46 mmol) and DIPEA (1.26 mL, 7.38 mmol) in DMF (20 mL) and the RM is stirred for 30 min. The RM is partitioned between water and EtOAc and the layers are separated. The aq. phase is re-extracted with EtOAc (2×) and the combined org. extracts are washed with brine, dried (Na₂SO₄), filtered, and evaporated. The crude product is purified by prep. HPLC (basic) to give benzyl 6-(((6S,9R,12R)-12-benzyl-6-isobutyl-2,2,5,8-tetramethyl-4,7,10-trioxo-9-(2,2,2-trifluoroethyl)-3-oxa-5,8,11-triazatridecan-13-yl)oxy)-3-fluoroquinoline-5-carboxylate as a white solid. LC-MS I: t_{R}=1.45 min; [M+H]⁺=811.73. Note: A 2nd stereoisomer, benzyl 6-(((6S,9S,12R)-12-benzyl-6-isobutyl-2,2,5,8-tetramethyl-4,7,10-trioxo-9-(2,2,2-trifluoroethyl)-3-oxa-5,8,11-triazatridecan-13-yl)oxy)-3-fluoroquinoline-5-carboxylate is also isolated as a white solid. LC-MS I: t_{R}=1.43 min; [M+H]⁺=811.66.

Step 2: A soln. of benzyl 6-(((6S,9R,12R)-12-benzyl-6-isobutyl-2,2,5,8-tetramethyl-4,7,10-trioxo-9-(2,2,2-trifluoroethyl)-3-oxa-5,8,11-triazatridecan-13-yl)oxy)-3-fluoroquinoline-5-carboxylate (519 mg, 0.61 mmol) in EtOH (10 mL) is evacuated/purged with N₂ (3×) before 10% Pd/C (32 mg, 5 mol %) is added. The RM is evacuated/purged with H₂ (3×) and stirred under a H₂ atm for 2 h. The RM is filtered through a pad of celite and the filtrate concentrated in vacuo to give 6-(((6S,9R,12R)-12-benzyl-6-isobutyl-2,2,5,8-tetramethyl-4,7,10-trioxo-9-(2,2,2-trifluoroethyl)-3-oxa-5,8,11-triazatridecan-13-yl)oxy)-3-fluoroquinoline-5-carboxylic acid as a white solid. LC-MS I: t_{R}=0.73 min; [M+H]⁺=721.58.

Step 3: HATU (417 mg, 1.09 mmol) is added to a RT soln. of 6-(((6S,9R,12R)-12-benzyl-6-isobutyl-2,2,5,8-tetramethyl-4,7,10-trioxo-9-(2,2,2-trifluoroethyl)-3-oxa-5,8,11-triazatridecan-13-yl)oxy)-3-fluoroquinoline-5-carboxylic acid (759 mg, 1.04 mmol), A-1 (390 mg, 1.04 mmol) and DIPEA, (0.55 mL, 3.13 mmol) in DMF (13 mL) and the RM is stirred for 1 h. The RM is partitioned between water and EtOAc and the layers are separated. The aq. phase is re-extracted with EtOAc (2×) and the combined org. extracts are washed successively with sat. aq. NaHCO₃, 1 M aq. citric acid, water, and brine. The org. extracts are dried (Na₂SO₄), filtered, and evaporated to give tert-butyl (S)-3-(6-(((6S,9R,12R)-12-benzyl-6-isobutyl-2,2,5,8-tetramethyl-4,7,10-trioxo-9-(2,2,2-trifluoroethyl)-3-oxa-5,8,11-triazatridecan-13-yl)oxy)-3-fluoroquinoline-5-carboxamido)-4-((2-(3-methoxy-1,2,4-oxadiazol-5-yl)ethyl)amino)-4-oxobutanoate as a white solid. LC-MS I: t_{R}=1.32 min; [M+H]⁺=1018.01.

Step 4: TFA (3.3 mL, 42.8 mmol) is added to a RT soln. of tert-butyl (S)-3-(6-(((6S,9R,12R)-12-benzyl-6-isobutyl-2,2,5,8-tetramethyl-4,7,10-trioxo-9-(2,2,2-trifluoroethyl)-3-oxa-5,8,11-triazatridecan-13-yl)oxy)-3-fluoroquinoline-5-carboxamido)-4-((2-(3-methoxy-1,2,4-oxadiazol-5-yl)ethyl)amino)-4-oxobutanoate (1.19 g, 1.1 mmol) in DCM (35 mL) and the RM is stirred for 3 h. The RM is concentrated in vacuo and the residue is re-dissolved in DCM and again concentrated in vacuo (2×). The residue is dissolved in DMF (18 mL) before DIPEA (1.51 mL, 8.8 mmol) and HATU (502 mg, 1.32 mmol) are added and the RM is stirred for 15 min. The RM is partitioned between water and EtOAc and the layers are separated. The aq. phase is re-extracted with EtOAc (2×) and the combined org. extracts are washed with brine, dried (Na₂SO₄), filtered, and evaporated. The crude product is purified by prep. HPLC (basic) to give the title compound as a white solid. LC-MS I: t_{R}=1.05 min; [M+H]⁺=843.68.

### II. BIOLOGICAL ASSAYS

### In Vitro Assay

The corrector activities of the compounds of formula (I) on CFTR are determined in accordance with the following experimental method. The method measures the effect of over-night compound incubation on F508del-CFTR cell surface expression in a recombinant U2OS cell line (DiscoveRx, #93-0987C3). This cell line is engineered to co-express (i) human F508del-CFTR tagged with a Prolink (PK=short β-galactosidase fragment) and (ii) the remainder of the β-galactosidase enzyme (Enzyme Acceptor; EA) localized to the plasma membrane. Incubation with compounds that increase PK-tagged F508del-CFTR at the plasma membrane will lead to complementation of the EA fragment to form a functional β-galactosidase enzyme which is quantified by a chemiluminescence reaction.

Briefly, the cells are seeded at 3500 cells/well into 384-well low volume plates (Corning, #3826) in 20 μl of full medium (Mc Coy's 5a (#36600-021, Gibco)+10% FBS Gibco+penicillin/streptomycin). The cells are incubated for 5 h in the incubator before the addition of 5 μl/well of compound dilution series (5× working stocks in full medium). Final DMSO concentration in the assay is 0.25%. The cells are co-incubated with the compounds for 16 h in the incubator at 37° C., 5% CO₂. The next day, the cell plates are incubated for 2 h at RT in the dark. Then, 10 μl/well of Flash detection reagent (DiscoverX, #93-0247) is added, the plate is incubated for another 30 min at RT in the dark and chemiluminescence is measured. Concentration-response curves are generated using compound-intrinsic maximal efficacy as upper plateau, and from these CRCs compound-intrinsic EC₅₀ values are determined. Compound-specific Eₘₐₓ values are calculated in relation to the Eₘₐₓ of the corrector lumacaftor (Eₘₐₓ lumacaftor=100%).

The calculated EC₅₀ values may fluctuate depending on the daily assay performance. Fluctuations of this kind are known to those skilled in the art. EC₅₀ values from several measurements are given as geomean values. The calculated Eₘₐₓ values may fluctuate depending on the daily assay performance. Fluctuations of this kind are known to those skilled in the art. Eₘₐₓ values from several measurements are given as arithmetic mean values.

The compound of Reference Example 1 was tested to have an EC₅₀ of 146 nmol/L and Eₘₐₓ of 460% in this assay.

### REFERENCES

Elborn J S. (2016) Cystic fibrosis. Lancet 388:2519-2531. Dalemans W, Barbry P, Champigny G, Jallat S, Dott K, Dreyer D, Crystal R G, Pavirani A, Lecocq J P, Lazdunski M (1991) Altered chloride ion channel kinetics associated with the delta F508 cystic fibrosis mutation. Nature 354: 526-8.

### III. EXAMPLES

### Example 1: Crystallization from an EtOH-Based Formulation

241 mg amorphous COMPOUND were dissolved in 3 mL of a mixture of ethanol, glyceryl monolinoleate, propyleneglycol and glycerol polyethylene glycol oxystearate (weight ratio 10%/36%/9%/45%). The solution turned into a suspension during overnight stirring at room temperature. The solid was filtered off and analyzed by XRPD (method 2), confirming the presence of crystalline material. The diffractogram was assigned to crystalline form 1 of the COMPOUND.

### Example 2: Crystallization from EtOH

10 mg of amorphous COMPOUND were suspended in 20 μL EtOH. The sample was stored in a closed vial at ambient temperature for 2 weeks. The solvent had evaporated after that time and the remaining solid was analyzed by XRPD (method 2), confirming the presence of the crystalline form 1 of the COMPOUND.

### Example 3: Crystallization from Toluene

10 mg of amorphous COMPOUND were dissolved in 20 μL toluene. The sample was stored in a closed vial at ambient temperature for 2 weeks. A solid had precipitated after that time and was analyzed by XRPD (method 2), confirming the presence of crystalline form 1 of the COMPOUND.

### Example 4: Crystallization from tBME with Seeding

10 mg of amorphous COMPOUND were dissolved in 50 μL tBME. Seeds of the product from example 1 were added and the mixture was agitated at room temperature. A white solid had formed after 5 d. It was confirmed to consist of form 1 of the COMPOUND by XRPD (method 2). TGA of the sample demonstrated the loss of 1.5% water in the temperature range from 50° C. to 160° C.

### Example 5: Crystallization from Heptane with Seeding

10 mg of amorphous COMPOUND were suspended in 50 μL heptane. Seeds of the product from example 1 were added and the mixture was agitated at room temperature. A white solid had formed after 12 d. It was confirmed to consist of form 1 of the COMPOUND by XRPD (method 2). TGA of the sample demonstrated the loss of 1.2% water in the temperature range from 50° C. to 150° C.

### Example 6: Crystallization from EtOH

0.5 mL EtOH were added to 100 mg of amorphous COMPOUND. While the original solid was still dissolving, a new solid formed, which was filtered off on the same day and dried under vacuum for 10 min, yielding 70 mg of solid. XRPD (method 1) confirmed that the solid consist of form 1 of the COMPOUND. TGA demonstrated the loss of 2.5% (0.5 equivalents) of EtOH between ca. 50° C. and 140° C. DSC exhibits a first broad endotherm between ca. 40° C. and 140° C., which is attributed to the loss of solvent. A sharp m.p. follows at 198° C. with an enthalpy of 41 J/g. SEM shows the presence of small needles and prisms with maximum lengths below 100 μm. GVS shows an almost linear change of the water content by 2% between 0% and 95% RH, a small hysteresis is observed between ca. 50% and 85% RH (see FIG. 4). The maximum water content of 2% corresponds to approximately 1 equivalent of water. After the GVS measurement, no residual EtOH was detected in the sample by TGA.

### Example 7: Crystallization from MeOH

A solution of 114 mg of COMPOUND in 1 mL MeOH was evaporated at ambient conditions. Hexagonal plates with dimensions up to several mm crystallized. Single crystal x-ray structure determination at 301 K elucidates an orthorhombic crystal system with the Hermann-Mauguin space group P 2₁ 2₁ 2₁ and the following unit cell parameters: a=14.4261(4) Å, b=22.1403(7) Å, c=27.0500(10) Å, α=90°, β=90°, γ=90°, V=8639.7(5) Å³, Z=8. The crystal structure determination demonstrates that one position in the crystal is partially occupied by methanol and water. The X-ray powder pattern that is calculated from the single crystal data is in good agreement with the experimentally determined pattern of the crystalline form 1.

### Example 8: Crystallization from EtOH/tBME with Seeding

7 g of amorphous COMPOUND were dissolved in 35 mL EtOH. Seeds of crystalline form 1 were added, which resulted in a thick suspension within 3 h. 25 mL tBME were added and stirring was continued for 1 h before cooling the mixture to 5° C. The solid was filtered off 30 min later, washed with 5 mL tBME and allowed to dry on the filter under normal atmosphere. 6.2 g of a white powder were obtained. XRPD (method 1) confirmed the crystallization of form 1 of the COMPOUND (see FIG. 1). A weight loss of 2.1% of EtOH (corresponding to ca. 0.4 equivalents) between 40° C. and 140° C. was observed by TGA. DSC showed a m.p. at 198° C. with an enthalpy of 45 J/g. HPLC indicates a purity of 99.85% a/a.

1. A crystalline form of (3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide 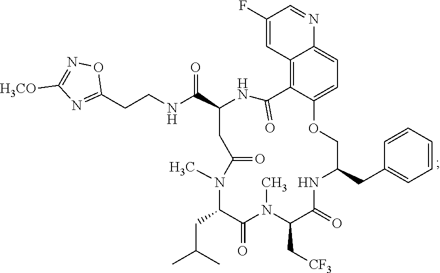 [SMILES: COc1noc(CCNC(=O)[C@@H]2CC(=O)N(C)[C@@H](CC(C)C)C(=O)N(C)[C@H](CC(F)(F)F)C(=O)N[C@H](Cc3ccccc3)COc3ccc4ncc(F)cc4c3C(=O)N2)n1]
characterized by the presence of at least four, or at least six, or at least eight peaks in the X-ray powder diffraction diagram at angles of refraction 2θselected from: 5.1°, 8.0°, 10.3°, 12.4°, 18.3°, 18.8°, 19.0°, 19.6°, 21.2°, and 22.0°.

2. A crystalline form of (3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide according to claim 1 characterized by the presence of peaks in the X-ray powder diffraction diagram at the following angles of refraction 2θ: 5.1°, 8.0°, and 18.8°.

3. A crystalline form of the compound (3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide according to claim 1, characterized by the presence of peaks in the X-ray powder diffraction diagram at the following angles of refraction 2θ: 5.1°, 8.0°, 10.3°, 18.8°, and 19.6°.

4. A crystalline form of the compound (3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide according to claim 1, characterized by the presence of peaks in the X-ray powder diffraction diagram at the following angles of refraction 2θ: 5.1°, 8.0°, 10.3°, 12.4°, 18.3°, 18.8°, 19.0°, 19.6°, 21.2°, and 22.0°.

5. A crystalline form of the compound (3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide according to claim 1, which essentially shows the X-ray powder diffraction pattern as depicted in FIG. 1.

6. A crystalline form of the compound (3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide according to claim 1, which exhibits an endothermal event at about 198° C. as determined by differential scanning calorimetry.

7. (canceled)

8. A pharmaceutical composition comprising as active ingredient a crystalline form of the compound (3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide according to claim 1, and at least one pharmaceutically acceptable carrier.

9. A process for preparing a pharmaceutical composition comprising (3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide wherein said process comprises mixing a crystalline form of the compound (3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide according to claim 1, and at least one pharmaceutically acceptable carrier material.

10-13. (canceled)

14. A method of treatment of CFTR-related diseases and disorders; comprising administering to a patient an effective amount of a crystalline form of the compound (3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide according to claim 1.

15. A method of treatment of cystic fibrosis; comprising administering to a patient an effective amount of a crystalline form of the compound (3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide according to claim 1.

16. A pharmaceutical composition comprising as active ingredient a crystalline form of the compound (3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide according to claim 2, and at least one pharmaceutically acceptable carrier.

17. A pharmaceutical composition comprising as active ingredient a crystalline form of the compound (3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide according to claim 3, and at least one pharmaceutically acceptable carrier.

18. A pharmaceutical composition comprising as active ingredient a crystalline form of the compound (3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide according to claim 4, and at least one pharmaceutically acceptable carrier.

19. A method of treatment of cystic fibrosis; comprising administering to a patient an effective amount of a pharmaceutical composition comprising a crystalline form of the compound (3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide according to claim 2.

20. A method of treatment of cystic fibrosis; comprising administering to a patient an effective amount of a pharmaceutical composition comprising a crystalline form of the compound (3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide according to claim 3.

21. A method of treatment of cystic fibrosis; comprising administering to a patient an effective amount of a pharmaceutical composition comprising a crystalline form of the compound (3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide according to claim 4.
